(19) United States
(12) Patent Application Publication
(10) Pub. No.: US 2026/0130964 A1
(43) Pub. Date: May 14, 2026

(54) PHARMACEUTICAL COMPOSITION COMPRISING HIGH PAY LOAD GINGER EXTRACT GRANULES AND PROCESS FOR PREPARATION THEREOF

(71) Applicant: INDIA GLYCOLS LIMITED, NOIDA (IN)

(72) Inventors: BAIDYANATH MAHANTA, NOIDA (IN); J. B. KANDPAL, NOIDA (IN); PRAGYA BHARTIA BARWALE, NOIDA (IN)

(21) Appl. No.: 19/117,976

(22) PCT Filed: Oct. 5, 2023
(86) PCT No.: PCT/IB2023/060003

§ 371 Date: Apr. 9, 2025

(30) Foreign Application Priority Data
Oct. 20, 2022 (IN) 202211059906

(51) Int. Cl. A61K 36/9068; A61K 9/16; A61K 9/48; A61K 9/50; A61K 31/12

(52) U.S. Cl. CPC A61K 36/9068; A61K 9/1611; A61K 9/1617; A61K 9/1635; A61K 9/1652; A61K 9/485; A61K 9/4858; A61K 9/4866; A61K 9/5026; A61K 9/5047; A61K 31/12; A61K 2236/37

(57) ABSTRACT

The present invention relates to a sustained release pharmaceutical composition comprising ginger extract. Particularly, this invention relates to sustained release granules of ginger extract wherein the ginger extract contains high payload of gingerols and shogaol. More particularly, the present invention relates to a sustained release granules of ginger extract wherein the granules release the gingerol and shogaols in sustained release manner. The present invention further relates to the process of preparation of ginger extract from ginger rhizome using supercritical fluid extraction wherein the extract is having high payload of gingerols and shogaol.

### FIELD OF INVENTION

The present invention relates to a sustained release pharmaceutical composition comprising ginger extract. Particularly, this invention relates to sustained release granules of ginger extract wherein the ginger extract contains high payload of gingerols and shogaol. More particularly, the present invention relates to a sustained release granules of ginger extract wherein the granules releases the gingerol and shogaols in sustained release manner. The present invention further relates to the process of preparation of ginger extract from ginger rhizome using supercritical fluid extraction wherein the extract is having high payload of gingerols and shogaol and the same is converted into granules having high payload of bioactive gingerols and shogaol.

### BACKGROUND

Botanicals bear many health promoting activities which led to the increased interest on the use of botanicals of controlling many ailments. It led to the increased standardization of botanical products as these are plant derived and have very few adverse impacts on the health of consumers. The use of botanical extracts as food supplements, dietary supplements and nutraceuticals has increased the added interest on research in this area.

Though the consumption of botanical extracts is increasing in last few decades the efficacy of these bioactives due to their low bioavailability remains an area of concern for the researchers. Due to the lipophilic nature of these compounds and their low solubility is main factor behind the lower bioavailability of the bioactives. To overcome the low bioavailability of these bioactives in the recent times a lot of focus is put on the method of isolating these botanical active compounds and innovating the novel delivery technologies for higher efficacy.

Ginger having many health promoting activities has been propagated for its use as nutraceuticals. The ginger extract is being extensively used to elicit its anti-inflammatory and/or thermogenic effects. The general method of extraction and isolation is based on the use of non-polar solvents like hexane, pentane; moderately polar solvent, ethanol and ethanol water. Though, the non-polar solvent extract selective compounds like essential oil of ginger and phenolic i.e. shogaol and Gingerols. The residual solvent left over in the extract is a challenging and concerned area. The problem is partially resolved by the use of polar solvent like ethanol and ethanol water. However, the extraction efficiency of polar solvent is lower and further it leads to the co-extraction of other compounds like starch and carbohydrates with the Gingerols, leading to low content of bioactive gingerol and shogaol in the extract.

The extraction limitation is partially overcome with the advent of supercritical fluid carbon dioxide. The supercritical carbon dioxide at optimized temperature and pressure led to the extraction of high content of gingerol and shogaol in the extract. The process also leads to the higher content of gingerols and shogaol as compared to the solvent extract. Another benefit of extraction with supercritical fluid carbon dioxide is the absence of any solvent residues which are very detrimental to the human health. Further, the super critical fluid extraction could be optimized by properly adjusting the pressure and temperature. In the current invention the optimized condition for supercritical extraction of Ginger were 250-700 bar pressure and 35-90 degree celsius temperature. The current invention led to 25-35% gingerols in the extract as compared to 15-22% gingerols in solvent extraction. Further as discussed, once the bioactive is isolated; the next phase is to enrich the bioactive to higher content by liquid-liquid extraction and solid-liquid extraction and finally develop the novel delivery technologies for the supercritical carbon dioxide extract/enriched extract so that the bioactive isolated could be used in efficacious manner.

The bioactive isolated like other drug substances needs to be given to the patient normally through oral route. Therefore, the taste of bioactive and or any drug substance is very crucial for its acceptance by the consumers. In the recent times, the taste masking of bitter and pungent bioactives/drugs has been major parameter for its acceptance. Nutraceuticals, like other drugs, are administered orally in many dosage forms like capsules, tablets, granules and liquid forms like solutions or suspensions. Conventional tablets and capsules are meant to be swallowed whole, hence generally taste masking is not so much desired and in case of capsules the drug is enclosed within the tasteless gelatine shell. For the children and older people the difficulty in swallowing require additional delivery/intake methods. If the drug is of obnoxious, pungent and of bitter taste, the patient compliance decreases. Hence, taste masking is a very important factor to increase patient compliance. A lot of technologies are available not only for masking the unwanted taste of bitter drugs but also help to increase the palatability of formulation and patient compliance. In addition, to mask the taste of sustained release granules of bioactive drug molecule is another important arena where much work is being done. As per studies the bioactive molecules are metabolized very fast because of which their therapeutic effect could not be achieved. This principle applied for ginger extract as well. Like Ginger, many other bioactive molecules have been widely used as healthy food condiment and for home remedies in Indian system of Medicine and traditional Chinese medicine owing to its efficacy in combating many health supporting activities. However, the biological half-life of 6-gingerol (a principal pungent ingredient of ginger) is less than <2 hrs while taken orally. So, increasing the half life for improving therapeutic effect is major area of concern before scientists. As mentioned above the Gingerols and Shogaols have a very short plasma half-life. Standard 5 mg dose gets washed away from the body within 2 hrs. To meet the requirement of sufficient concentration in the plasma higher dose will be required. In addition to higher dose the stability of the gingerols and shogaol composition is very important for having its health benefits. Moreover, the chronic ailments such as joint pain and obesity require the Gingerols and Shogaols to be present in blood circulation for at least 10 hrs. To take care of chronic ailments the need of high payload gingerols and shogaol formulations having ginger sustained release granules 20-25 mg are developed which are currently not found commercially.

Improving bioavailability of gingerols by different formulation technologies and by targeted delivery, this problem could be solved to great extent. For achieving these objectives, the work attempted by different researchers/manufacturer is described below.

WO2017103946 A2 relates to a composition exhibiting enhanced bioavailability comprising tri-molecular complex of a natural compound or a natural compound containing component, a divalent or trivalent metal compound and a phospholipid embedded in the natural matrix.

WO2017221268 Aa relates to a taste masking formulations for bitter natural compounds, selected from the extracts, fraction and pure phytochemicals produced in combination with a synthetic polymers such as Eudragit or natural polymers such as Shellac.

None of these references describes sustained release with higher percentage of gingerols and Shogaols of ginger rhizome with effective controlled released bioactive in physiological pH to show efficacy for prolonged periods in small effective dosages.

U.S. Pat. No. 6,099,865 discloses a taste masking procedure for pharmaceutical drug substances using croscarmellose sodium, particularly the masking of bitter taste.

Korean Patent No. 101195528B1 discloses a ginger extract composition comprising crude oil extract as flavor controlling the flavor/taste and release of the product.

WO2003045411A2 discloses a method of extraction of stable ginger preparation. The ginger extract is stabilized with at least one stabilizing auxiliary agent selected from the group of proton-capturing substances, whereby the chemically stabilizing auxiliary agent preferably belongs to the family of polyvinylpyrrolidone and releases the included pungent agents in solution.

As discussed above the current literature on the taste masking and sustained release of bioactive of ginger a very scanty. This is because preparation of palatable sustained release formulation of pungent and bitter bioactive compound is more challenging as compared to the synthetic drugs substances. Due to the complexities involved in masking of taste and to controlled release; a few efforts has been made till now.

The taste masked and sustained release products of botanicals have additional inherent disadvantages which include lack of stability of the formulation resulting in reappearance of the bitter taste when the formulation is subjected to physical stress, dose formulation, liquid formulation, dissolution in liquid. In addition, these techniques affect the stability and bioavailability of the actives. The present invention overcomes the various drawbacks of the literature.

The present invention intend to develop a method to mask the pungent taste of the ginger extract along with the preparation of a sustained release product which will be therapeutically more effective. Further, the composition is developed with ingredients/excipients which are stable and converted to different doses form of capsules, tablets and pellets.

### OBJECTIVE OF THE INVENTION

The main objective of the present invention is to develop a process of preparation of ginger extract having high concentration of gingerols and shogaol from the ginger rhizome using supercritical fluid.

Another objective of the present invention is to develop a process of preparation of ginger extract from ginger rhizome using supercritical carbon dioxide.

Another objective of the present invention is to develop a process of preparation of ginger extract from ginger rhizome using supercritical carbon dioxide wherein the extract is having high content of bioactive gingerols.

Another objective of the present invention is to develop a process of enrichment of Gingerols and shogaols from 25-35% to 40-70% through liquid-liquid extraction, solid-liquid extraction, solvent purification and/or supercritical carbon dioxide enrichment process to get highest payload of gingerols.

Another objective of the present invention is to develop a sustained release composition comprising ginger extract and optionally one or more pharmaceutically/food grade acceptable excipients.

Another objective of the present invention is to develop a sustained release granule comprising ginger extract and optionally one or more pharmaceutically acceptable excipients.

Another objective of the present invention is to develop a sustained release dosage form comprising ginger extract and optionally one or more pharmaceutically acceptable excipients.

Another objective of the present invention is to develop a sustained release pharmaceutical composition comprising taste masked ginger extract and one or more pharmaceutically acceptable excipients.

Another objective of the present invention is to develop a sustained release taste masked pharmaceutical composition comprising ginger extract, a sustained release agent and one or more taste masking agents.

Another objective of the present invention is to develop a taste masked pharmaceutical composition comprising ginger extract and one or more taste masking agents.

### SUMMARY OF THE INVENTION

The present invention relates to a process of preparation of ginger extract from ginger rhizome.

The present invention further relates to a process of preparation of ginger extract from the ginger rhizome using supercritical fluid supercritical carbon dioxide at a pressure 350-450 bar and temperature 39-90 degree Celsius.

The present invention further relates to a process of preparation of ginger extract from ginger rhizome using supercritical carbon dioxide.

The present invention further relates to a process of preparation of ginger extract from ginger rhizome using supercritical carbon dioxide wherein the extract is having high payload of gingerol and shogaol.

The present invention relates to a process of preparation of ginger extract from ginger rhizome comprising the steps of:
a) Grating/cutting the ginger rhizome into smaller pieces;
b) Grinding the small pieces of ginger rhizome to powder form;
c) Conducting the supercritical carbon dioxide extraction of the powdered mass of ginger rhizome.

The present invention further relates to a process of preparation of ginger extract from ginger rhizome comprising the steps of:
a) Grating/cutting the ginger rhizome into smaller pieces.
b) Grinding the small pieces of ginger rhizome to powder form;
c) Treating the ginger rhizome with supercritical carbon dioxide;
d) Isolating the extract from supercritical carbon dioxide wherein the ginger extract is having the high payload of gingerol and shogaol.

The present invention further relates to a process of preparation of ginger extract from ginger rhizome comprising the steps of:
a) Grating/cutting the ginger rhizome into smaller pieces;
b) Grinding the small pieces of ginger rhizomes to powder form;
c) Treating the ginger rhizome with supercritical carbon dioxide;
d) Isolating the extract from supercritical carbon dioxide;
e) Further liquid-liquid extraction of the primary extraction with supercritical carbon dioxide solvent to prepare high payload of gingerols and shogaol in the extract.

The present invention further relates to a pharmaceutical composition comprising ginger extract and one or more pharmaceutically acceptable excipients.

The present invention further relates to a sustained release pharmaceutical composition comprising ginger extract and one or more pharmaceutically acceptable excipients.

The present invention further relates to a sustained release granules comprising ginger extract and one or more pharmaceutically acceptable excipients.

The present invention further relates to a taste masked and sustained release pharmaceutical composition comprising ginger extract and one or more taste masking agents like carnauba wax and bees wax.

The present invention further relates to a sustained release pharmaceutical composition comprising taste masked ginger extract and one or more pharmaceutically acceptable excipients.

### DESCRIPTION OF THE DRAWINGS

FIG. 1: Percent gingerol release in 1.2 pH buffer followed by 6.8 pH buffer with time.

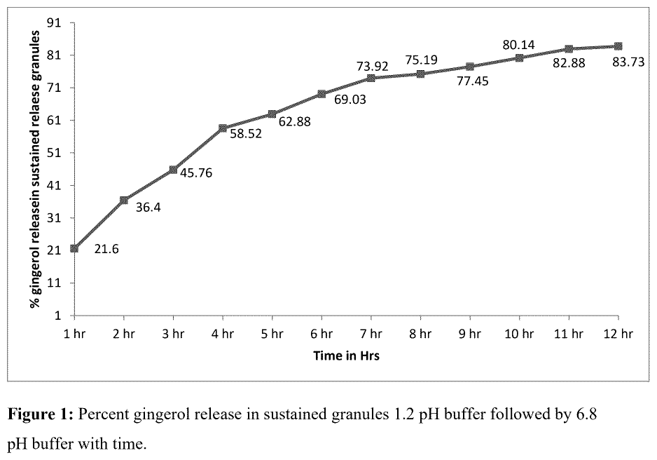

### DETAILED DESCRIPTION OF THE INVENTION

In one embodiment the present invention relates to a process of preparation of ginger extract from ginger rhizome.

In another embodiment the present invention relates to a process of preparation of ginger extract from the ginger rhizome using supercritical carbon dioxide.

In another embodiment the present invention relates to a process of preparation of ginger extract from ginger rhizome using supercritical carbon dioxide.

In another embodiment the present invention relates to a process of preparation of ginger extract from ginger rhizome comprising the steps of:
a) Grating/cutting the ginger rhizome into smaller pieces.
b) Grinding the small pieces of ginger rhizome to powder form;
c) Treating the ginger rhizome with supercritical fluid.

In another embodiment the present invention relates to a process of preparation of ginger extract from ginger rhizome comprising the steps of:
a) Grating/cutting the ginger rhizome into smaller pieces;
b) Grinding the small pieces of ginger rhizome to powder form;
c) Treating the ginger rhizome with supercritical fluid.
d) Isolating the extract from solvent.

The supercritical fluid according to the present invention is supercritical form of any of the solvent selected from the group consisting of carbon dioxide, water, methane, ethane, propane, ethylene, propylene, methanol, ethanol, acetone, nitrous oxide or combinations thereof. More specifically the supercritical fluid is supercritical carbon dioxide.

Grating or cutting the ginger rhizome into smaller pieces can be done using any cutting, grating or milling equipment such as knife, cutter, grater, multi-mill, ball mill or the like.

Grinding of the ginger rhizomes relates to the fine sizing of the ginger for better extraction during supercritical fluid extraction due to the higher diffusion thereby improving the extraction.

Isolation of the extract from the supercritical fluid can be done with the change in temperature and pressure after extraction. The transition of phase from liquid to gas can be done based on the removal of pressure over the liquid extract.

The extraction of gingerol and shogaol from ginger rhizome is done in the temperature range of about 35° C.-90° C., preferably in the range of about 50° C.-65° C.

In another embodiment the present invention relates to a process of preparation of ginger extract from ginger rhizome comprising the steps of:
a) Grating/cutting the ginger rhizome into smaller pieces;
b) Grinding the small pieces of ginger rhizomes to powder form;
c) Treating the ginger rhizome with supercritical carbon dioxide at a temperature in the range of 35° C.-90° C.;
d) Isolating the extract from solvent.

In another embodiment the present invention relates to a process of preparation of ginger extract from ginger rhizome comprising the steps of:
a) Grating/cutting the ginger rhizome into smaller pieces;
b) Grinding the small pieces of ginger rhizome to powder form;
c) Treating the ginger rhizome with supercritical carbon dioxide at a temperature in the range of 50° C.-65° C.;
d) Isolating the extract from solvent.

The extraction of gingerol and shogaol from ginger rhizome is done at the pressure in the range of about 200 to 800 bars, preferably in the range of about 250-700 bars.

In another embodiment the present invention relates to a process of preparation of ginger extract from ginger rhizome comprising the steps of:
a) Grating/cutting the ginger rhizome into smaller pieces;
b) Grinding the small pieces of ginger rhizomes to powder form;
c) Treating the ginger rhizome with supercritical carbon dioxide at a temperature in the range of 50° C.-65° C. and pressure in the range of 350-450 bar.
d) Isolating the extract from solvent.

Ginger extract obtained from the process according to present invention is having a high payload i.e. from 40 to 70% of gingerol and shogaol and more specifically to 50-60%.

The present invention focuses not only the taste masking with the application of wax coating/and or hydroxy propyl methyl cellulose coating. The high payload composition of gingerols and shogaols prepared with the carnauba wax, bees wax, plant waxes, calcium silicate and stearic acid as done in the current investigation led to sustained release composition of gingerols and shogaol which may be very useful for chronic ailments like obesity and joint pains. The masking of the taste will make the granules composition easier and acceptable besides the release in sustained manner which will not lead to the irritation or stomach pain to the consumers due to the high payload. The sustained release composition is targeted for its release so that continuous availability of gingerols for longer time after ingestion is ensured which is easily tolerable. Further the composition is developed with stable ingredients/excipients and converted to different dosage form of capsules, tablets and pellets.

In the present invention the product of gingerols is developed to address the issue of high pungency as well as sustained release for providing better efficacy. The composition developed in the current invention led to the masking of the taste and development of sustained release product.

The present invention release the drug at targeted sites and in controlled manner so as to achieve better therapeutic efficacy for longer duration of time.

The sustained release of gingerols will make it available for prolonged period for controlling the joint pain and obesity. Therefore the focus of the current invention is to prepare high payload ginger granules which are easily palatable as taste is masked and lead to sustained release at different time interval to combat the chronic ailments like joint pain and obesity. The present invention results in continuous availability of gingerols and shogaol for long period and that also in high concentration as the focus is on high payload of gingerols 20-35% to act effectively for chronic joint pain and obesity.

In another embodiment the present invention relates to a pharmaceutical composition comprising ginger extract and one or more pharmaceutically acceptable excipients.

The pharmaceutical composition according to the present invention contains about 5-40% of ginger extract, preferably about 25-35% more preferably 20-30% active gingerols from ginger extract.

In another embodiment the present invention relates to a sustained release pharmaceutical composition comprising ginger extract and one or more pharmaceutically acceptable excipients.

Sustained release pharmaceutical composition or granules or dosage form according to present invention is the one which releases the active ingredient slowly for an extended period of time. The time period of slow release can range from about 2 hours to 20 hours; preferably in the range from about 6 hours to about 16 hours more preferably, in the range from about 8 hours to about 12 hours.

Pharmaceutically acceptable excipients according to present invention include but not limited to diluents, binder, sustained release agent or release controlling agent, stabilizer, taste masking agent, lubricant, glidant, coating agent, pH regulating agent, antioxidant, dispersant etc.

Suitable stabilizer used according to the present invention are selected from but not limited to Carnauba wax, bees wax, stearic acid, plant waxes extracted from plants particularly sugar cane wax etc.

Suitable “diluents” used according to the present invention are selected from water soluble or water insoluble or combination thereof. Suitable water soluble diluents include sucrose, dextrose, lactose, mannitol, sorbitol and the like and water insoluble diluents include starch, microcrystalline cellulose, silicified microcrystalline cellulose, calcium silicate and the like or combination thereof. The amount of diluent may range from about 10% to 95% by weight.

The term “binders” as used herein means any material that is added to pharmaceutical compositions to help hold such compositions together and release the medicament therefrom. Binder can be selected from the group consisting of polymers (water soluble or water insoluble polymer), waxes (natural or others) such as but not limited to beeswax, sugar cane wax, carnauba wax, semisynthetic or synthetic plastic substances.

Polymeric binder can be selected from the group consisting of but not limited to, starch, pre-gelatinized starch, starch paste, povidone, sucrose, polyethylene glycol and/or polymers such as tragacanth, HPMC, ethyl cellulose, hydroxypropyl cellulose, carboxymethylcellulose, maltodextrin, starch ester, acacia, gelatin, cellulose, polymethacrylates, polyvinyl alcohols, polyvinyl pyrrolidone, maltodextrin, Beta cyclodextrin, starch ester acetate, and combinations thereof.

The binder according to present invention can be used in the range from about 5-70%; preferably between about 20-50% and more preferably between about 10-35%.

The taste masking agent can be selected from the group comprising ion exchange resin, cyclodextrins, organic acids such as tartaric, fumaric and the like, bases such as sodium bicarbonate, sodium carbonate and the like; maltodextrin and starch esters.

Suitable disintegrants used according to the present invention are selected from starch, crosspovidone, sodium starch glycolate, croscarmellose sodium and the like or combination thereof. The amount of disintegrant may range from about 1% to 10% by weight.

Suitable lubricants used according to the present invention are selected from magnesium stearate, hydrogenated castor oil, calcium stearate, sodium stearyl fumarate, talc, vegetable oils, stearic acid, fumaric acid and the like.

The term “glidants” as used herein refers to a substance, such as an anti-caking agent, which improves the flow characteristics of a powder mixture.

Suitable glidants include silicon dioxide and talc. 0.5 to 1%.

Suitable film coating polymers used according to the present invention are selected from povidone, ethylcellulose, hydroxypropylcellulose, hydroxypropylmethyl cellulose, polyvinyl alcohol and the like or mixtures thereof. The amount of polymer may range from about 0.001 to 5.0 w/v %.

The current invention to prepare sustained release formulation of ginger focuses on utilize the waxes and thermoplastic substances which have melting point above 50 degree Celsius, More preferably between 50 degree Celsius and below 130 degree Celsius, more preferably below 70-90 degree Celsius. The potential binder substances used for the current invention contain higher chain fatty alcohols and fatty esters and are generally water insoluble compounds. In addition the invention focuses on the use of hydrophobic water insoluble wax like substances like bees wax, carnauba wax, Plant derived waxes and microcrystalline wax etc.

The additional substances/excipients/fillers for the formulation ranges from calcium silicate, calcium hydroxide, calcium carbonate, magnesium carbonate, calcium hydrogen phosphate, magnesium stearate, liquid glucose, microcrystalline cellulose, hydroxypropyl methyl cellulose may also be used for coating of granules. The fillers used for preparing the granules of present invention will not only act as buffer but also control the release rate of bioactive agent.

The present invention further relates to a sustained release granules comprising ginger extract and one or more pharmaceutically acceptable excipients.

The release of drug from the granules according to the present invention may be controlled by varying the size of the granules. The smaller granule size will have larger surface area and will affect the release of active from the granules. The present invention of preparation of ginger granules do not totally control the burst effect however the same is overcome by controlling the surface area of granules by making smaller granules. The current invention focuses on granules pass to 20 mesh and retained by 30 mesh sieve. The granules with 20 mesh sieve pass showed sustained release during dissolution trials.

The present invention further relates to a taste masked pharmaceutical composition comprising ginger extract and one or more taste masking agents.

The present invention further relates to a sustained release pharmaceutical composition comprising taste masked ginger extract and one or more pharmaceutically acceptable excipients.

The pharmaceutical composition according to present invention can be used to prepare any dosage form such as granules, minitablets, pellets, tablet, capsule, transdermal or any other oral dosage form.

The granules prepared in the current invention are to be used orally and on ingestion will pass to gastrointestinal passage. Therefore, the pH environment of ingested pellets changes during the passage. Because of this change, the solubility of the drug particles may change as well as the solubility and stability of the filler and binder matrix. The effect of the varying solubility during passage through the gastrointestinal tract can be countered by addition of auxiliary substances having an acid or a basic character contributing to a buffered “micro-environment” in the inventive pellets. To control the release of gingerol from the dosage form according to present invention basic substances like calcium hydroxide/silicate and acidic substances like stearic acid has been used.

The selection between a basic and an acidic buffering substance will control the release profile as well as the stability of granules.

In another embodiment the sustain release pharmaceutical composition comprising:
i) Ginger oleoresin
ii) Stearic acid
iii) Carnauba Wax
iv) Calcium Silicate

In another embodiment the sustain release pharmaceutical composition comprising:
i) 5 to 35.0% of active component Ginger rhizome extract-Ginger oleoresin containing gingerols and shogaols (20.0% to 70.0%) along with resin and essential oils
ii) 5.0 to 50.0% of Carnauba wax or bees wax
iii) 5.0 to 50.0% of stearic acid
iv) 1.0 to 30.0% of calcium silicate
v) 1.0 to 20.0% of di-calcium phosphate
vi) 2.0 to 30.0% of hydroxyl propyl methylcellulose
vii) 1.0 to 15.0% of magnesium stearate

In another embodiment the present invention relates to a process of preparing a sustained release pharmaceutical composition comprising ginger extract and one or more pharmaceutically acceptable excipients.

The pharmaceutical composition according to present invention can be prepared using any conventional method such as wet granulation, dry granulation, compaction, melt granulation, pelletisation, complexation, solid dispersion, extrusion-spheronisation, hot melt extrusion and the like.

Further to prepare the ginger granules having sustained release properties with the above composition is derived from Gingerols and Shogaols (5 to 35%), taste masking of developed formulation along with sustained release in GIT for prolong period for 8 hour to 15 hour. The targeted formulation of the invention will have better health supplementing potentials due to the higher concentration achieved in blood plasma for longer duration.

For preparing the sustained release product the ginger oleoresin containing gingerols and shogaol is mixed with calcium salt like calcium silicate. The adsorbed mass is sieved with 30 mesh. The wax particularly carnauba wax, bees wax, sugar cane press mud wax and stearic acid are taken together and melted at a temperature ranging from 80 to 130 degree celsius avoiding the charring of the admixture. The molten mass of carnauba wax and stearic acid is mixed with ginger oleoresin adsorbed in calcium silicate. The mixture is granulated in a granulator and pass through the sieves ranging from 20-40 mesh.

The ginger granules prepared with the method described according to present invention shows complete masking of pungent taste. The solubility of Ginger granules is performed in different pH buffer like (1.4, 4.5 and 6.8) and dissolution profile is observed in pH 1.4 (1 hr) and pH 6.8 (1 hr to 16 hr).

The sustained release ginger composition as per the present invention is prepared with ginger oleoresin having high payload of 64% gingerols by adding carnauba wax and stearic acid. The composition developed contain high payload of 24.2% gingerols in the granules.

In another embodiment the 68% gingerols used for the preparation of high payload sustained release ginger granules and spray coating in Fluid bed processor with 1-2% hydroxypropyl methylcellulose.

In another embodiment the high payload ginger granules prepared with a coating of hydroxypropyl methyl cellulose are dried by fluidization at a temperature 25-30 degree celsius.

In another embodiment ginger oleoresin 50% are used for the preparation of high payload ginger granules containing high payload of 25.6% gingerols.

In another embodiment bees wax and carnauba wax used in combination with stearic acid for the preparation of high payload sustained release ginger granules.

In another embodiment high content of Ginger oleoresin with 51.92% gingerols and shogaol is used for the preparation of high payload sustained release ginger granules.

In another embodiment sustained release ginger granules are prepared from high payload ginger oleoresin containing 50% gingerols and final granules having 23.4% gingerols which will be suitable for preparation of doses form capsules, tablets and pellets.

In another embodiment the dissolution of high payload ginger granules was carried out in dissolution apparatus maintaining 50 RPM at a temperature 35 degree celsius. The release of Gingerols was observed in 0.1N Hydrochloric acid pH 1.2 for 1 h and 2h respectively. Total volume of the dissolution media is kept 900 ml.

The dissolution was further studied in Phosphate buffer pH 6.8 for 1 h, 2h, 3 h, 4 h, 5 h, 6 h, 7 h, 8 h, 9 h and finally for 10 h. The overall release for acidic pH 1.2 and pH 6.8 was studied for 12 h release.

The Gingerols and shogaol analysis for the release of sustained release granules was conducted by HPLC-Shimadzu Nexera-XR

The detailed analysis parameters mentioned as below.

### HPLC Parameter

Mobile phase: 0.1% OPA in water and ACN, (35:65)
Diluent: MeOH
Run time 40 min
Wave length 282 nm
Column C18 150 mm×4.6, 5 micron
Flow: 0.6 ml/min
Column temperature: 25° C.
Injection volume: 20 μL

The high payload sustained release granules as studied in another embodiment the dissolution of 37% gingerols at a pH of 1.2 in two hours.

In another embodiment the ginger granules after resulted in atleast 70% release at 12 hours.

In another embodiment the ginger granules after 37% release in acidic pH resulted in total 78% release at 12 hours in pH 6.8 phosphate buffer.

In another embodiment the ginger granules led to 44% release in first 2 hours and overall 80% release in 12 hours including the acidic and pH 6.8.

Further the method of preparation of ginger granules of present invention resulted in stable sustained release of active constituent gingerol further the formulation developed contain high payload of gingerols ranging from 5-35%. In one of the embodiments of the invention, provides the composition and manufacturing process of said formulation.

### Example 1: Extraction of Ginger Oleoresin from Ginger Rhizome

### Example 1a: Preparation of Ginger Extract with More than 27% Gingerol

3 kg of Ground Ginger rhizome was filled in a supercritical carbon dioxide extraction basket. Supercritical carbon dioxide was passed through it and extraction was conducted at 350-600 bar and 50-70 degree celsius temperature. Supercritical carbon dioxide was passed at a rate of 1.0 kg/minute. Supercritical carbon dioxide was passed at 60 kg/per kgs of biomass and at the end of extraction the ginger extract was collected from separator 1 and 2. The primary separator had gingerol content 27% and in separator-2 the gingerol content was 20%.

### Assay by HPLC:

### HPLC Conditions:

Column: C18 (150× 4.6 mm) 5 μm
Flow Rate: 0.6 ml/min
Wavelength: 282 nm.
Mobile Phase: 0.1% Orthophosphoric acid: Acetonitrile (35:65).

### Retention Times:

| | |
|---|---|
| 6 Gingerol | about 4.3 min. |
| 8 Gingerol | about 7.0 min |
| 10 Gingerol | about 14.0 min |
| 6 Shogaol | about 8.5 min |
| Stop Time: | 40 min. |

### Preparation of Standard Solution:

Weigh about 40 mg of Gingerol standard (40%) and dissolve in 100 ml methanol. Filter and inject.

### Preparation of Sample Solution:

Weigh about 150 mg of sample and dissolve in 100 ml methanol, sonicate. Filter and inject.

### Injection Sequence:

| | |
|---|---|
| Blank | 1 run |
| Standard | 3 run |
| Sample | 2 run |
| Standard | 1 run |

### System Suitability:

1. % RSD of gingerols and shogaol from first three run of the standard should be not more than 2.0%.
2. % RSD of 6-gingerols by including bracketing standard and omitting first run of standard should be not more than 2.0%.

### Calculation:

$$Assay = \frac{Average Sample area}{Average Standard area} \times \frac{Dilution of Standard}{Dilution of Sample} \times Standard Potency$$

Calculate each Gingerol and Shogaol by putting individual peak area and potency.

Finally calculate total gingerols as:

$$Total Gingerols = ( 6 - gingerol + 8 - gingerol + 10 - gingerol + 6 - shogaol )$$

$$Gingerols = ( 6 - gingerol + 8 - gingerol + 10 - gingerol )$$

### Example 1b: Preparation of Ginger Extract with More than 35% Gingerol

3.5 kgs of ground Ginger rhizome was filled in a supercritical carbon dioxide extraction basket. Supercritical carbon dioxide was passed through it and extraction was conducted at 400-500 bar and 50-65 degree celsius temperature. Supercritical carbon dioxide was passed at a rate of 1.0 kg/minute. Supercritical carbon dioxide was passed at 65 kg/per kgs of biomass and at the end of extraction the ginger extract was collected from separator 1 and 2. The primary separator had gingerol content 35% and in separator-2 the gingerol content was 23%.

### Example 1c: Preparation of Ginger Extract with More than 50% Gingerol

Ginger rhizome extract from the separator −1 and Separator −02 was charged in the liquid-liquid extractor for super critical carbon dioxide extraction. Super critical carbon dioxide was passed at a flow rate of 0.6 kg/minute at 150-200 bar. The gingerols were extracted and the reextracted mass was collected. The pressure was increased to 250-300 bar to extract remaining gingerols. The content of gingerols in separator−01 was 65% and separator −02 was 43%. The gingerextract of two separators was mixed to get 51.92% gingerols and was used for preparation of high payload sustained release ginger granules.

### Example 1d

The ginger rhizome extract of the previous examples were charged for extraction of gingerol in the liquid-liquid extractor for super critical carbon dioxide extraction. Super critical carbon dioxide was passed at a flow rate of 0.7 kg/minute at 180-225-200. The gingerols were re-extracted and collected. The pressure was further increased to 250-325 bar to extract remaining gingerols. The content of gingerols in separator-01 was 68% and separator −02 was 40%. The separator −01 ginger extract having high payload of Gingerols 68% is used for preparation of high payload sustained release ginger granules.

### Example 2

| S. No. | Ingredients | Quantity in % w/w |
|---|---|---|
| 1 | Ginger oleoresin | 21.32% |
| 2 | Dicalcium phosphate | 1.96% |
| 3 | Hydroxyl propyl methyl cellulose | 53.2% |
| 4 | Starch | 3.72% |
| 5 | Xanthan gum | 16.0% |
| 6 | Polyvinylpyrrolidone | 2.18% |
| 7 | Silicon dioxide | 1.08% |
| 8 | Magnesium stearate | 1.08% |
| 9 | water | q.s. |

### Examples-3 to 6

| | | Exam- | Exam- | Exam- | Exam- |
|---|---|---|---|---|---|
| | | ple 3 | ple4 | ple5 | ple 6 |
| S. No. | Ingredients | Quantity in % w/w | | | |
| 1 | Ginger oleoresin | 30.0% | 40.0% | 50.0% | 75.0% |
| 2 | Stearic acid | 9.8% | 9.4% | 5.0% | 2.8% |
| 3 | Carnauba wax | 40.2% | 25.8% | 17.8% | 10.0% |
| 4 | Calcium silicate | 20.0% | 24.8% | 27.2% | 12.2% |

The composition of Examples 3 to 6 was prepared according to the process as follows:
a) Ginger oleoresin was heated in a mixing vessel.
b) Carnauba wax and stearic acid were mixed and heated in the separate vessel to get the wax in molten state.
c) Added the heated ginger oleoresin of step 1 to the molten carnauba wax and mixed the same under mild heat.
d) Added microcrystalline cellulose to the mixture of step 3 and granulated by adding water.
e) The entire mass was mixed and dried in vacuum dryer at 20-25 degree celsius under vacuum. Sifted the dried mass through 20 mesh sieve to get Ginger granules.

### Examples 7 to 13

| S. | | Ex. 7 | Ex. 8 | Ex. 9 | Ex. 10 | Ex. 11 | Ex. 12 | Ex. 13 |
|---|---|---|---|---|---|---|---|---|
| No. | Ingredients | % w/w | % w/w | % w/w | % w/w | % w/w | % w/w | % w/w |
| | Gingerol % | 64% | 68% | 52% | 68% | 51.92% | 52% | 51.92% |
| 1 | Ginger | 33.74 | 32.34 | 52.70 | 41.27 | 44.49 | 44.80 | 45.70 |
| | oleoresin | | | | | | | |
| 2 | Carnauba | 21.63 | 26.35 | 16.38 | 9.60 | 6.36 | 20.00 | 19.05 |
| | wax | | | | | | | |
| | Bees wax | — | — | — | 4.80 | — | — | — |
| 3 | Stearic | 2.25 | 4.00 | 3.14 | 17.27 | 19.07 | 3.20 | 4.76 |
| | acid | | | | | | | |
| 4 | Calcium | 28.54 | 24.35 | | 15.36 | 16.94 | 21.20 | 19.05 |
| | silicate | | | | | | | |
| 5 | Hydroxypropyl | — | 12.97 | 27.78 | 3.84 | 4.24 | 10.80 | 11.44 |
| | methyl | | | | | | | |
| | cellulose | | | | | | | |
| 6 | Microcrystalline | 13.84 | — | — | — | — | — | — |
| | cellulose | | | | | | | |
| 7. | Ethyl | — | — | — | 7.87 | 8.90 | — | — |
| | cellulose | | | | | | | |
| | Total | 100 | 100 | 100 | 100 | 100 | 100 | 100 |

### Example 7

390 g of ginger oleoresin (64%) was taken in a mixing vessel and heated. 330 g of calcium silicate was added in the above mixing vessel. 250 g of carnauba wax and 26 g of stearic acid was taken separately in another vessel and heated to get the wax in molten state. The heated ginger oleoresin was added to the molten carnauba wax and mixed under mild heat. Finally to this uniformly mixed mass added 160 g of microcrystalline cellulose and granulated by adding water. Mixed the entire mass and dried in vacuum dryer at 20-25 degree celsius under vacuum. The dried mass was taken out and sifted the same through 20 mesh sieve to get ginger granules 930 g containing 24.2% Gingerols.

### Example 8

162 g of ginger oleoresin (68%) and 122 g calcium silicate was taken in mixing vessel and heated to get molten mass. 132 g carnauba wax and 20 g stearic acid was taken in separate vessel and heated to get molten product. Ginger oleoresin containing calcium silicate molten mass was added to the molten carnauba wax stearic acid mixture to prepare matrix for granulation. The above mass was mixed thoroughly to get uniform mixture. 65 g of hydroxypropyl methyl cellulose was added in the above mixture. The above mass was granulated by adding/sprinkling water. The mass was sifted through 20 mesh sieve and dried the sifted mass at a temperature 20-25 degree celsius under vacuum. 490 gm of the granules were unloaded containing 21.4% gingerols.

### Example 9

370 g of ginger oleoresin (52%) was taken in a mixing vessel and heated. 115 g of carnauba wax and 22 g stearic acid was taken in separate vessel and heated the mixture to get molten mass. The mixture of ginger oleoresin was added to the molten mixture of carnauba wax and stearic acid and mixed uniformly. 195 g of hydroxypropyl methyl cellulose was added to the above mixture and mixed under heating. 100-125 ml water was sprinkled to granulate the mass. The mixed mass was dried in a vacuum oven to get the dried mass and sifted through 20 mesh sieve to get ginger granules 680 g containing 25.6% gingerols.

### Example 10

100 g carnauba wax, 50 g bees wax and 180 g stearic acid was taken in a mixing vessel and mixed under heating to get molten mass. 430 g of Ginger oleoresin containing 68% gingerols and 160 g calcium silicate was taken in separate vessel and mixed well. The ginger oleoresin containing calcium silicate was transferred to the molten carnauba wax, bees wax and stearic acid molten mass. To this mass added 70 g ethyl cellulose and 30 g hydroxypropyl methyl cellulose and heated to make uniform mixture under mixing/blending. Mixture of 12 g ethyl cellulose and 10 g hydroxypropyl methyl cellulose was prepared in water (2% solution) and sprayed this mixture to coat the prepared dried ginger matrix. The granules were sifted through 20 mesh sieve and dried under constant heat 20 degree celsius in fluid bed processor. The granules were unloaded and weighed the same to get 940 g ginger granules having 28.5% gingerol content.

### Example 11

210 g of ginger oleoresin containing 51.92% Gingerols and 80 g calcium silicate was taken in a vessel and heated to make uniform mass. To this mass added 90 g stearic acid and 30 g carnauba wax. Heated the mixed mass to get molten mixture and to this mass added 42 g ethyl cellulose and 20 g hydroxypropyl methyl cellulose in 50 g water. Mixed uniformly and heated the same. The mixed mass was taken out and dried under vacuum to get 450 g ginger granules containing 21.8% gingerols.

### Example 12

560 g (52% Gingerols) ginger oleoresin was taken in a mixing vessel and added 265 g of calcium and heat to get the molten mass. Separately weighed 250 g of carnauba wax and 40 g of stearic acid and heated to melt. The melted gingerol and calcium silicate mixture was transferred to the carnauba wax, stearic acid molten mass. The entire mass was mixed properly and transferred to a fluid bed processor and coated with an aqueous mass of 135 g hydroxypropyl methyl cellulose in 200 ml water. Dried the mass under fluidization and sifted the same to get 1100 g ginger granules containing 24.8% Gingerols.

### Example 13

240 g of ginger oleoresin (51.92% gingerols) and 100 g calcium silicate was taken in a heated vessel to get molten mass. 100 g of carnauba wax and 25 g stearic acid was heated to get molten mass in a separate vessel. The ginger oleoresin, calcium silicate molten mass was added to the melted carnauba wax and stearic acid mixture. The matrix obtained from above was coated with 60 g hydroxypropyl methyl cellulose. Dried the mass under vacuum to get dried granules and sifted to get 460 g ginger granules containing 23.4% Gingerols.

### Example 14: Dissolution Study of Ginger Granules

200 mg ginger granules were taken in a dissolution apparatus and the dissolution was observed at pH 1.2 for one and two hours. The granules were then transferred to another vessel and the dissolution in phosphate buffer pH 6.8 was observed. The release in acidic pH in 2 hours was 37% and further in 8 hour at pH 6.8 the 78% gingerols release was observed. The results showed the sustained release effect of granules in acidic and basic pH of 1.2 and 6.8 respectively. The sustained release profile of ginger granules for four experiments is presented in FIGS. 6 and 7 respectively.

1. A composition comprising high payload of Gingerol for joint pain and obesity comprising:
i) ginger extract containing about 40% to 70% Gingerols;
ii) one or more stabilizing agent;
iii) one or more pharmaceutical/food grade excipient and
iv) optionally a film forming polymer.

2. The composition according to claim 1 wherein the stabilizing agent is selected from the group consisting of carnauba wax, bees wax, plant derived waxes or combinations thereof.

3. The composition according to claim 1 wherein the pharmaceutical/food grade excipient is selected from the group consisting of diluents, binder, lubricant, glidant, disintegrant, taste masking agent and film forming agent.

4. The composition according to claim 1 wherein the composition is formulated as granules, pellets, tablets, capsules, suspension, gel or combinations thereof.

5. The composition according to claim 1 wherein the composition is a sustained release granule.

6. The composition according to claim 1 wherein the composition comprises 20-35% gingerol and shogaol.

7. The composition according to claim 1 wherein the ginger extract with high gingerol content is prepared by supercritical carbon dioxide extraction of ginger rhizome at 250-700 bar pressure and 35-90 degree celsius temperature.

8. The composition according to claim 1 wherein the binder is selected from the group consisting of hydroxypropyl methyl cellulose, methyl cellulose, carboxymethyl cellulose, hydroxypropyl cellulose, maltodextrin, Beta cyclodextrin, starch ester acetate, polyvinyl alcohol, polyvinyl pyrrolidone either alone or in combination thereof.

9. The composition according to claim 1 wherein the composition comprises ginger oleoresin extract from about 21% to about 75%, carnauba wax from about 10 to about 40%, stearic acid from about 5% to about 9.8% and calcium silicate from about 12% to about 27.2% and optionally magnesium stearate.

10. The composition according to claim 1 wherein the composition further comprises sustained release agent selected from group consisting of ethyl cellulose, hydroxypropyl methyl cellulose, methyl cellulose, carboxymethyl cellulose, hydroxypropyl cellulose or combinations thereof; and taste masking agent selected from ion exchange resin, cyclodextrins, organic acids, bases, maltodextrin and starch esters.
